(19) United States
(12) Patent Application Publication
(10) Pub. No.: US 2022/0261636 A1
(43) Pub. Date: Aug. 18, 2022

(54) MACHINE LEARNING DEVELOPMENT USING SUFFICIENTLY-LABELED DATA

(71) Applicant: University of Florida Research Foundation, Incorporated, Gainesville, FL (US)

(72) Inventors: Jose C. Principe, Gainesville, FL (US); Shiyu Duan, Cupertino, CA (US)

(21) Appl. No.: 17/584,505

(22) Filed: Jan. 26, 2022

Related U.S. Application Data
(60) Provisional application No. 63/200,120, filed on Feb. 16, 2021.

(51) Int. Cl. G06N 3/08; G06K 9/62; G16H 50/30

(52) U.S. Cl. CPC G06N 3/08; G16H 50/30; G06K 9/628; G06K 9/6256

(57) ABSTRACT

Embodiments of the present disclosure provide methods, apparatus, systems, computing devices, computing entities, and/or the like for training a machine learning model comprising a hidden module and an output module and configured for identifying one of a plurality of original labels for an input. In accordance with one embodiment, a method is provided that includes generating sufficiently-labeled data comprising example-pairs each associated with a sufficient label. The sufficient label of an example-pair indicates whether a first and a second input example have the same original label. The method further includes training the hidden module using the sufficiently-labeled data, and subsequently, training the output module using a plurality of input examples each having an original label. The plurality of input examples may be a plurality of fully-labeled data. The method further includes automatically providing the resulting trained machine learning model for use in prediction tasks.

### CROSS-REFERENCE TO RELATED APPLICATIONS

This application claims priority to U.S. Provisional Patent Application Ser. No. 63/200,120 filed on Feb. 16, 2021, which is incorporated herein by reference in its entirety, including any figures, tables, drawings, and appendices.

### GOVERNMENT SUPPORT

This invention was made with government support under FA9453-18-1-0039 awarded by the US Department of Defense DARPA. The government has certain rights in the invention.

### TECHNOLOGICAL FIELD

Embodiments of the present disclosure generally relate to a technology framework for developing and/or training machine learning models without the need for supplying labels for every training example as conventionally done.

### BACKGROUND

In supervised learning, obtaining a large set of fully-labeled training data for developing and/or training machine learning models can be expensive, time consuming, and inefficient. Accordingly, a need exists in the industry to address technical challenges related to providing processes and/or systems for efficiently and/or directly obtaining training data that captures sufficient label information relevant for developing and training machine learning models, without necessarily the need to collect and/or use labels for the full set of the data. It is with respect to these considerations and others that the disclosure herein is presented.

### BRIEF SUMMARY

In accordance with one aspect of the present disclosure, a method for training a machine learning model including a hidden module and an output module and configured for predicting one of a plurality of original labels for an input is provided. The method includes generating sufficiently-labeled data including a plurality of example-pairs. Each example-pair is associated with a sufficient label indicating whether a first input example and a second input example of the example-pair are identified as having a same original label from the plurality of original labels. The method further includes training the hidden module of the machine learning model using the sufficiently-labeled data. The method further includes, sequentially after the training of the hidden module of the machine learning model, training the output module of the machine learning model using a plurality of input examples each having one of the plurality of original labels to generate a trained machine learning model. The method further includes automatically providing the trained machine learning model for use in one or more prediction tasks.

In accordance with another aspect of the present disclosure, an apparatus for training a machine learning model including a hidden module and an output module and configured for predicting one of a plurality of original labels for an input is provided. The apparatus includes at least one processor and at least one memory including program code. The at least one memory and the program code are configured to, with the at least one processor, cause the apparatus to generate sufficiently-labeled data including a plurality of example-pairs. Each example-pair is associated with a sufficient label indicating whether a first input example and a second input example of the example-pair are identified as having a same original label from the plurality of original labels. The at least one memory and the program code are further configured to, with the at least one processor, cause the apparatus to train the hidden module of the machine learning model using the sufficiently-labeled data. The at least one memory and the program code are further configured to, with the at least one processor, cause the apparatus to, sequentially after the training of the hidden module of the machine learning model, train the output module of the machine learning model using a plurality of input examples each having one of the plurality of original labels to generate a trained machine learning model. The at least one memory and the program code are further configured to, with the at least one processor, cause the apparatus to automatically provide the trained machine learning model for use in one or more prediction tasks.

In accordance with another aspect of the present disclosure, a non-transitory computer storage medium for training a machine learning model including a hidden module and an output module and configured for predicting one of a plurality of original labels for an input is provided. The non-transitory computer storage medium includes instructions configured to cause one or more processors to at least perform operations configured to generate sufficiently-labeled data including a plurality of example-pairs. Each example-pair is associated with a sufficient label indicating whether a first input example and a second input example of the example-pair are identified as having a same original label from the plurality of original labels. The non-transitory computer storage medium further includes instructions configured to cause one or more processors to at least perform operations configured to train the hidden module of the machine learning model using the sufficiently-labeled data. The non-transitory computer storage medium further includes instructions configured to cause one or more processors to at least perform operations configured to, sequentially after the training of the hidden module of the machine learning model, train the output module of the machine learning model using a plurality of input examples each having one of the plurality of original labels to generate a trained machine learning model. The non-transitory computer storage medium further includes instructions configured to cause one or more processors to at least perform operations configured to automatically provide the trained machine learning model for use in one or more prediction tasks.

### BRIEF DESCRIPTION OF THE DRAWING(S)

Having thus described the disclosure in general terms, reference will now be made to the accompanying drawings, which are not necessarily drawn to scale, and wherein:

FIG. 1 is a diagram of a system architecture that can be used in conjunction with various embodiments of the present disclosure;

FIG. 2 is a schematic of a computing entity that may be used in conjunction with various embodiments of the present disclosure;

FIG. 3 is a process flow for obtaining and using training data for developing and/or training a machine learning model in accordance with various embodiments of the present disclosure;

FIG. 4 provides an example of using sufficiently-labeled data as a layer of encryption for sensitive data in accordance with various embodiments of the present disclosure;

FIG. 5 is a process flow for training a machine learning model in accordance with various embodiments of the present disclosure; and

FIG. 6 is an example of a learning pattern in a feature space based on sufficiently-labeled data in accordance with various embodiments of the present disclosure.

### DETAILED DESCRIPTION OF SOME EXAMPLE EMBODIMENTS

Various embodiments of the present disclosure now will be described more fully hereinafter with reference to the accompanying drawings, in which some, but not all embodiments of the disclosure are shown. Indeed, the disclosure may be embodied in many different forms and should not be construed as limited to the embodiments set forth herein; rather, these embodiments are provided so that this disclosure will satisfy applicable legal requirements. The term “or” (also designated as “/”) is used herein in both the alternative and conjunctive sense, unless otherwise indicated. The terms “illustrative” and “exemplary” are used to be examples with no indication of quality level. Like numbers refer to like elements throughout.

### Overview

Embodiments of the disclosure provide a novel framework for developing and/or training machine learning models without the need for using a set of training data that has been fully-labeled. Accordingly, various embodiments of the disclosure are described herein with respect to developing and/or training a machine learning model by using supervised learning with improved efficiency. Supervised learning is generally understood to be a machine learning task involving the model learning a function that maps an input to an output based on explicit label information for each input (e.g., the explicit label information being fully labeled data, in some examples). Here, according to various embodiments of the present disclosure, machine learning models are configured to infer the same function by employing class partnership to all pairs of inputs (both examples belong to the same class or not), i.e. the sufficient labels, and only requiring full labels for a minimal subset of inputs.

For instance, consider a machine learning model that involves a multiclass (e.g., c-class) classification task with independent and identically distributed data {Xᵢ, Yᵢ, Yᵢ}ᵢ₌₁ⁿ, with each Xᵢ being an input example and Yᵢ∈{1, . . . , c} its label. Conventional training of such a machine learning model is normally conducted in which every single training example used for estimating a competent classifier for the model is fully labeled. However, various embodiments of the disclosure presented herein provide a novel framework that enables training of a machine learning model without the need to have every single training example used for training the model under a supervised learning approach to be fully labeled.

Accordingly, various embodiments of disclosure involve extending and applying statistical sufficiency principles in providing a framework that can be used in developing and/or training machine learning models with a focus on reducing the need and/or cost of obtaining labeled training data. On a high level, a sufficiency statistic can be viewed as a function of data that comprises all its information when it comes to estimating an unknown parameter of the underlying distribution. Therefore, various embodiments of the disclosure provide a framework that makes use of such a function on fully-labeled data, referred to herein as sufficiently-labeled data, in developing and/or training machine learning models. As described further herein, in particular embodiments, the sufficiently-labeled data can be obtained directly without having to collect fully-labeled data first and/or can be more easily obtained compared to fully-labeled data, and at the same time capture relevant information from data for learning optimal hidden representations.

For instance, in particular embodiments, the framework may be configured for developing and/or training a machine learning model having a hidden module and an output module. For example, such models may include neural networks and/or kernel machines, although other mappings constructed by a composition of functions may be included. In general, the hidden module is configured for mapping an input representation (space) to a feature representation (space). For example, in some embodiments, the hidden module may include a plurality of neural network layers, or other form of machine learning models configured to map into a high-dimensional feature space such as a layer of kernel machines. While in some embodiments, the output module may be a linear model in said feature space.

In these particular embodiments, the framework may be configured for training the hidden module using sufficiently-labeled data to obtain learned hidden representations. The framework may be configured to then train the output module on the learned hidden representations using a set of fully-labeled data. In some embodiments, the set of fully-labeled data needed in training the output module to attain a particular level of performance may include a reduced number of examples than the number of examples needed in a set of fully-labeled data used in training the model to attain the same level of performance under a conventional framework. For instance, in some embodiments, the framework is able to train the output module involving a classifier using as few as a single randomly-chosen fully-labeled example per class. Thus, an advantage provided by the framework in various embodiments is the allowance of learn performant models with less costly training data through the use of sufficiently-labeled data in place of fully-labeled data.

In addition, another advantage provided by the framework in various embodiments is the use of sufficiently-labeled data that is naturally suitable for privacy-preserving learning. Specifically, in particular embodiments, the framework may be configured to use a set of sufficiently-labeled training data that contains only “relative labels” on user pairs, in which no information on individuals can be re-identified based on these relative labels. This can be ideal in settings where the labels to be predicted by the model on individuals contain sensitive information such as, for example, a diagnosis result for an individual of a certain disease. Accordingly, some embodiments of the framework may facilitate having sensitive user information that no longer need to be stored and/or transported, thereby enhancing the security and privacy of the communication and/or storage pipeline without extra overhead or compromise in performance.

Accordingly, various embodiments of the framework, as described further herein, make the following contributions. As noted, embodiments of the framework make use of a novel function of data referred to as sufficiently-labeled data having input example-pairs in developing and/or training machine learning models. In particular embodiments, each example-pair found in the set of sufficiently-labeled data may have a binary label stating whether the two examples for a pair are from the same class, or are from different classes. Accordingly, various embodiments of the disclosure provide a framework for developing and/or training machine learning models in which the models learn with a mixture of sufficiently-labeled and fully-labeled data. As discussed further herein, in particular embodiments, a hidden module of a model is trained using sufficiently-labeled data, and an output module is then trained using fully-labeled data without fine-tuning the hidden module. Accordingly, in various embodiments, the sufficiently-labeled data is sufficient for finding the optimal hidden module parameters and as a result, the framework can produce solutions that are as competent as those produced using fully-labeled data with comparable sample complexity. In addition, in particular embodiments, having more sufficiently-labeled data can reduce the need for fully-labeled data. Finally, in various embodiments, sufficiently-labeled data may be derived from fully-labeled data, but it may also be directly collected with the advantage of privacy, and/or may be easier to collect than fully-labeled data. Further detail is now provided on different aspects of the framework accordingly to various embodiments of the disclosure.

### Computer Program Products, Systems, Methods, and Computing Entities

Embodiments of the present disclosure may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises a combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially, such that one instruction is retrieved, loaded, and executed at a time. In some exemplary embodiments, retrieval, loading, and/or execution may be performed in parallel, such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### Exemplary System Architecture

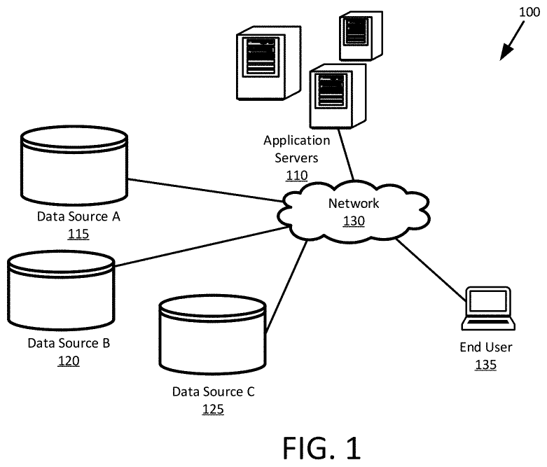

FIG. 1 provides an illustration of a system architecture 100 that may be used in accordance with various embodiments of the disclosure. Here, the architecture 100 includes various components involved in training and/or using a machine learning model in accordance with various embodiments. Accordingly, the components may include one or more application servers 110 that may be in communication with one or more data sources 115, 120, 125 over one or more networks 130. It should be understood that the application server(s) 110 may be made up of several servers, storage media, layers, and/or other components, which may be chained or otherwise configured to interact and/or perform tasks. Specifically, the application server(s) 110 may include any appropriate hardware and/or software for interacting with the data sources 115, 120, 125 as needed to execute aspects of one or more applications for processing data acquired from the data sources 115, 120, 125 and handling data access and business logic for such.

In addition, the architecture 100 may include one or more computing devices 135 used by end users for conducting one or more processes involving training and/or making use of a machine learning model configured in accordance with various embodiments of the disclosure. Here, the device(s) 135 may be one of many different types of devices such as, for example, a desktop or laptop computer or a mobile device such as a smart phone or tablet.

As noted, the application server(s) 110, data sources 115, 120, 125, and computing device(s) 135 may communicate with one another over one or more networks 130. Depending on the embodiment, these networks 130 may comprise any type of known network such as a land area network (LAN), wireless land area network (WLAN), wide area network (WAN), metropolitan area network (MAN), wireless communication network, the Internet, etc., or combination thereof. In addition, these networks 130 may comprise any combination of standard communication technologies and protocols. For example, communications may be carried over the networks 130 by link technologies such as Ethernet, 802.11, CDMA, 3G, 4G, or digital subscriber line (DSL). Further, the networks 130 may support a plurality of networking protocols, including the hypertext transfer protocol (HTTP), the transmission control protocol/internet protocol (TCP/IP), or the file transfer protocol (FTP), and the data transferred over the networks 130 may be encrypted using technologies such as, for example, transport layer security (TLS), secure sockets layer (SSL), and internet protocol security (IPsec). Those skilled in the art will recognize FIG. 1 represents but one possible configuration of a system architecture 100, and that variations are possible with respect to the protocols, facilities, components, technologies, and equipment used.

### Exemplary Computing Entity

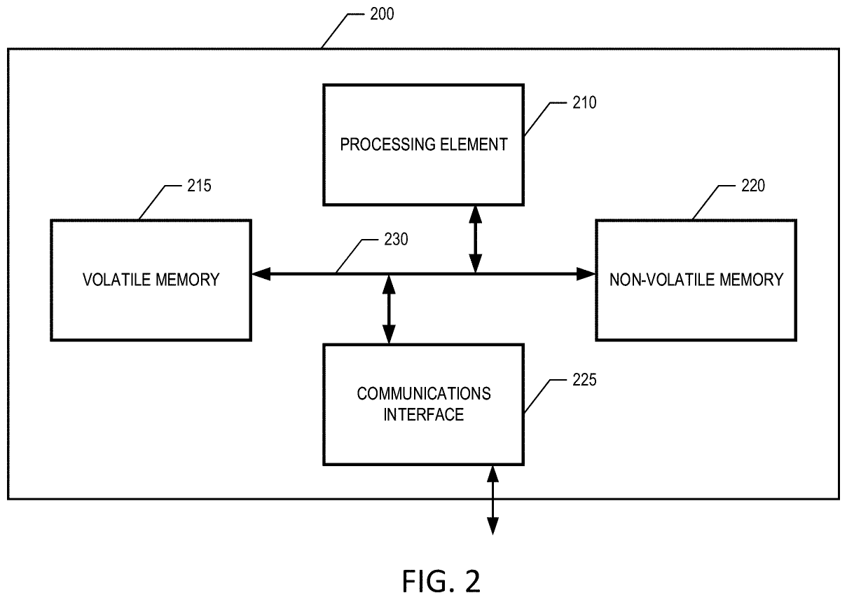

FIG. 2 provides a schematic of a computing entity 200 that may be used in accordance with various embodiments of the present disclosure. For instance, the computing entity 200 may be embodied by one or more of the application servers 110, and/or one or more of the computing devices 135, previously described in FIG. 1. In general, the terms computing entity, entity, device, system, and/or similar words used herein interchangeably may refer to, for example, one or more computers, computing entities, desktop computers, mobile phones, tablets, phablets, notebooks, laptops, distributed systems, items/devices, terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

Although illustrated as a single computing entity, those of ordinary skill in the art should appreciate that the computing entity 200 shown in FIG. 2 may be embodied as a plurality of computing entities, tools, and/or the like operating collectively to perform one or more processes, methods, and/or steps. As just one non-limiting example, the computing entity 200 may comprise a plurality of individual data tools, each of which may perform specified tasks and/or processes.

Depending on the embodiment, the computing entity 200 may include one or more network and/or communications interfaces 225 for communicating with various computing entities, such as by communicating data, content, information, and/or similar terms used herein interchangeably that can be transmitted, received, operated on, processed, displayed, stored, and/or the like. Thus, in certain embodiments, the computing entity 200 may be configured to receive data from one or more data sources and/or devices as well as receive data indicative of input, for example, from a device.

The networks used for communicating may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks may have any suitable communication range associated therewith and may include, for example, global networks (e.g., the Internet), MANs, WANs, LANs, or PANs. In addition, the networks may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms provided by network providers or other entities.

Accordingly, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the computing entity 200 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol. The computing entity 200 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

In addition, in various embodiments, the computing entity 200 includes or is in communication with one or more processing elements 210 (also referred to as processors, processing circuitry, and/or similar terms used herein interchangeably) that communicate with other elements within the computing entity 200 via a bus 230, for example, or network connection. As will be understood, the processing element 210 may be embodied in several different ways. For example, the processing element 210 may be embodied as one or more complex programmable logic devices (CPLDs), microprocessors, multi-core processors, coprocessing entities, application-specific instruction-set processors (ASIPs), and/or controllers. Further, the processing element 210 may be embodied as one or more other processing devices or circuitry. The term circuitry may refer to an entirely hardware embodiment or a combination of hardware and computer program products. Thus, the processing element 210 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 210 may be configured for a particular use or configured to execute instructions stored in volatile or non-volatile media or otherwise accessible to the processing element 210. As such, whether configured by hardware, computer program products, or a combination thereof, the processing element 210 may be capable of performing steps or operations according to embodiments of the present disclosure when configured accordingly.

In various embodiments, the computing entity 200 may include or be in communication with non-volatile media (also referred to as non-volatile storage, memory, memory storage, memory circuitry and/or similar terms used herein interchangeably). For instance, the non-volatile storage or memory may include one or more non-volatile storage or memory media 220, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or memory media 220 may store files, databases, database instances, database management system entities, images, data, applications, programs, program modules, scripts, source code, object code, byte code, compiled code, interpreted code, machine code, executable instructions, and/or the like. The term database, database instance, database management system entity, and/or similar terms used herein interchangeably and, in a general sense, to refer to a structured or unstructured collection of information/data that is stored in a computer-readable storage medium.

In particular embodiments, the memory media 220 may also be embodied as a data storage device or devices, as a separate database server or servers, or as a combination of data storage devices and separate database servers. Further, in some embodiments, the memory media 220 may be embodied as a distributed repository such that some of the stored information/data is stored centrally in a location within the system and other information/data is stored in one or more remote locations. Alternatively, in some embodiments, the distributed repository may be distributed over a plurality of remote storage locations only. As already discussed, various embodiments contemplated herein communicate with various information sources and/or devices in which some or all the information/data required for various embodiments of the disclosure may be stored.

In various embodiments, the computing entity 200 may further include or be in communication with volatile media (also referred to as volatile storage, memory, memory storage, memory circuitry and/or similar terms used herein interchangeably). For instance, the volatile storage or memory may also include one or more volatile storage or memory media 215 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory media 215 may be used to store at least portions of the databases, database instances, database management system entities, data, images, applications, programs, program modules, scripts, source code, object code, byte code, compiled code, interpreted code, machine code, executable instructions, and/or the like being executed by, for example, the processing element 210. Thus, the databases, database instances, database management system entities, data, images, applications, programs, program modules, scripts, source code, object code, byte code, compiled code, interpreted code, machine code, executable instructions, and/or the like may be used to control certain aspects of the operation of the computing entity 200 with the assistance of the processing element 210 and operating system.

As will be appreciated, one or more of the computing entity's components may be located remotely from other computing entity components, such as in a distributed system. Furthermore, one or more of the components may be aggregated and additional components performing functions described herein may be included in the computing entity 200. Thus, the computing entity 200 can be adapted to accommodate a variety of needs and circumstances.

### Exemplary System Operations

The logical operations described herein may be implemented (1) as a sequence of computer implemented acts or one or more program modules and/or applications running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, modules, or applications. These states, operations, structural devices, acts, modules, and applications may be implemented in software, in firmware, in special purpose digital logic, and any combination thereof. Greater or fewer operations may be performed than shown in the figures and described herein. These operations may also be performed in a different order than those described herein.

### Exemplary Machine Learning Model Development

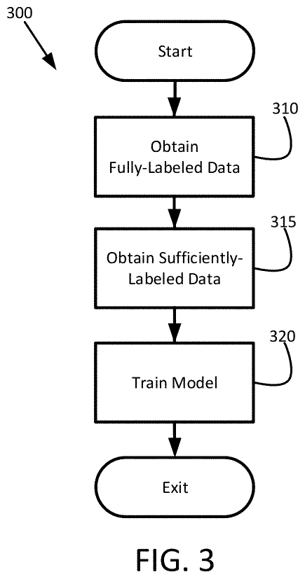

Turning now to FIG. 3, additional details are provided regarding a process flow for developing a machine learning model according to various embodiments. For instance, the process flow may involve the development of a machine learning model to be used in the context of a finance or healthcare application. For example, a health insurance provider may be developing the machine learning model to use in predicting the likelihood of insured individuals in developing a medical condition, such as cancer, based on the individuals' medical histories. Here, the machine learning model may be configured as a binary classifier (two class) machine learning model, such as, for example, a neural network, with a hidden module and an output module. In various embodiments, steps/operations of the process flow shown in FIG. 3 may be performed by the computing entity 200. For example, as the computing entity 200 may be embodied by application servers 110 and/or an end user device 135, as shown in FIG. 1.

For example, the machine learning model may be a two-class classification model with data being random elements X∈ [symbol] ⊆ [symbol] ^{d}, Y∈{+, −}, where X is the input and Y its label, and [symbol] is a subspace. A density function (X, Y) for the inputs and labels may be represented by p_{X,Y}(x, y). For any two independent inputs and their corresponding labels (e.g., (X₁, Y₁), (X₂Y₂)), Equation 1 may describe their density function.

p_{X}_{₁}_{,X}_{₂}_{|Y}_{₁}_{,Y}_{₂}(x₁,x₂)=p_{X}_{₁}_{|Y}(x₁)p_{X}_{₂}_{|Y}(x₂)

### Equation 1

Accordingly, the process flow 300 shown in FIG. 3 for training the machine learning model may begin with obtaining a set of fully-labeled data to be used in training the model in Step/Operation 310. As previously noted, the set of fully-labeled data is used in various embodiments to train the output module of the model. Therefore, depending the embodiment, the set of fully-labeled data may include a reduced/limited number of examples in comparison to the number of examples found in a set of fully-labeled data used for training a model under a conventional approach.

For instance, returning to the example in which a health insurance provider is developing a machine learning model to predicting the likelihood of insured individuals in developing a medical condition, the set of fully-labeled data may include examples representing insured individuals. Here, each example may identify an original label as to whether the insured individual represented by the example developed the medical condition or not. In addition, the example may include data that may be provided as input to the machine learning model in training the output module of the model. Therefore, each example may be considered a combination of an input and output, with the output being the original label for the example.

In various embodiments, the fully-labeled data may be retrieved, received, accessed, and/or the like from a data source, such as the data sources 115, 120, 125 shown in FIG. 1. For example, at Step/Operation 310, the computing entity 200 receives the fully-labeled data over a network 130. In other example embodiments, the computing entity 200 may locally store and retrieve the fully-labeled data, and in some examples, the fully-labeled data may originate from user input at the computing entity 200 and/or other computing devices.

Next, a set of sufficiently-labeled data is obtained in Step/Operation 315. Given random variables Y, Y′, a sufficient label can be defined by Equation 2.

T(Y,Y′)= [symbol] (Y=Y′).

### Equation 2

In Equation 2, [symbol] represents the indicator function. The support of a function or random element f is denoted supp f The cardinality of a set [symbol] is written as | [symbol] |. Accordingly, a set of sufficiently-labeled data of size n∈ [symbol] can be given by Equation 3.

{(X₁_{ᵢ},X₂_{ᵢ},T(Y₁_{ᵢ},Y₂_{ᵢ}))}ᵢ₌₁ⁿ

### Equation 3

In Equation 3, {(Xⱼ_{ᵢ}, Yⱼ_{ᵢ})}_{j=1,2,i=1, . . . , n} represents a set of independent and identically) distributed random elements sharing distribution with (X, Y). As previously discussed, the set of sufficiently-labeled data is used in various embodiments for training the hidden module of the machine learning model.

Accordingly, in various embodiments, the set of sufficiently-labeled data may be derived from fully-labeled data. That is, at Step/Operation 315, obtaining the sufficiently-labeled data comprises generating or deriving the sufficiently-labeled data from the fully-labeled data. In particular embodiments, each example-pair found in the sufficiently-labeled data may be derived from summarizing a set of two examples found in the fully-labeled data. Thus, an example-pair of the sufficiently-labeled data may be derived and may generally describe two examples or samples from the fully-labeled data. For instance, in some embodiments, a sufficient label may be a summary of the original labels on a set of fully-labeled examples of size two (X₁, Y₁), (X₂, Y₂) found in the fully-labeled data. Specifically, in some embodiments, the sufficient label is binary and summarizes whether the two examples that make up the example-pair are from the same class or from different classes. For instance, the two examples may be labeled using integers to represent classes, or some type of distinct symbols to represent classes that may be encoded into integers. Thus, in various embodiments, each sufficiently-labeled example-pair found in the set of sufficiently-labeled data may be derived by reducing a set of fully-labeled examples {(Xᵢ, Yᵢ)}ᵢ₌₁²ⁿ into a sufficiently-labeled example-pair that is a summary of the original set of examples, as described by Equation 4.

{(X₁_{ᵢ},X₂_{ᵢ},T(Y₁_{ᵢ},Y₂_{ᵢ}))}ᵢ₌₁ⁿ

### Equation 4

Accordingly, depending on the embodiment, the reduction from fully-labeled data to sufficiently-labeled data may be performed in multiple ways by arranging the indicates 1ᵢ, 2ᵢ. In various embodiments, the sufficiently-labeled data may include example-pairs for each unique pairing of data samples or examples found in the fully-labeled data.

For instance, in particular embodiments, the example-pairs can be generated from a set of fully-labeled single examples by performing arbitrary pairwise combinations. For example, the fully-labeled examples may be patient records. Here, a set of sufficiently-labeled data: {(X1, X2, T(Y1, Y2), (X1, X3, T(Y1, Y3), (X2, X3), T(Y2, Y3)} can be produced from a set of three fully-labeled examples {(X1, Y1), (X2, Y2), (X3, Y3)}, wherein T is defined as above in Equation 2.

In other embodiments, the set of sufficiently-labeled data may be obtained from data that is not necessarily fully-labeled. For instance, in particular embodiments, an end user (e.g., an annotator) may label example-pairs for the set of sufficiently-labeled data as to whether each example-pair is associated with the same original label such as, for example, whether each example-pair is from the same class (1) or not (0), without having to specify exactly the class partnership of each exemplar. In other embodiments, a computational model may be used in automatically assigning sufficient labels to example-pairs. For example, an annotation machine learning model may be developed and trained to identify raw data from the example-pairs and assign sufficient labels to the example-pairs accordingly based at least in part on the annotation machine learning model predicting whether the paired examples belong to the same class, generally. For instance, the annotation machine learning model may be configured using a sufficient classifier that is trained to produce sufficient labels on the example-pairs. In some embodiments, the actual/original label of each individual example found in a sample pair does not need to be identified. Therefore, as a result, less effort on the part of end users, as well as computational capacity on the part of a computing entity running a model, may be required in obtaining the set of sufficiently-labeled data in particular embodiments than if each individual example needed to be fully labeled.

In addition, determining whether the examples in a given example-pair are associated with the same label (e.g., are from the same class) is simpler for an annotator and/or model compared to determining a specific label (e.g., a specific class) of each of the examples in the pair. Moreover, sufficiently-label data is simpler to obtain in various embodiments than fully-labeled data. Specifically, learning to produce full labels for unlabeled examples can have a larger sample complexity than learning to produce sufficient labels for unlabeled example-pairs according to various embodiments as the number of underlying labels (e.g., classes) increases. As a result, training a competent model to assign sufficient labels for example-pairs for various embodiments can often require fewer labeled training examples than training a competent model to assign full labels.

To demonstrate such, the Gaussian complexity of a multi-class (c-class) classification problem using common loss functions such as the hinge loss or cross-entropy loss is [symbol] (c²/\sqrt{n}) in general, where n is the labeled training sample size. However, generating sufficient labels in various embodiments is a two-class classification problem regardless of the number of actual classes. Therefore, the sample complexity of generating sufficient labels in these embodiments is [symbol] (1/\sqrt{n}), whereas the sample complexity for generating full labels is [symbol] (c²/\sqrt{n}). Therefore, learning to produce full labels for unlabeled examples has a larger sample complexity than learning to produce sufficient labels for unlabeled example-pairs in various embodiments. As result, in particular embodiments, training a competent model to label example-pairs for a set of sufficiently-labeled data can require fewer training examples than training a model to label examples for a set of fully-labeled data. This is generally true, regardless of the machine learning model that is to be trained using the set of sufficiently-labeled data.

Once the appropriate training data has been obtained, the process flow 300 continues with training the machine learning model in Step/Operation 320. In various embodiments, the training of the machine learning model is carried out by a training application. Accordingly, as discussed further herein, the training application is configured in these embodiments to conduct the training of the machine learning model based on a framework that makes use of the two sets of data: the set of sufficiently-labeled data and the set of fully-labeled data.

Accordingly, once the training of the model has been completed, the result is a trained version of the machine learning model that may then be used in processing unseen input to identify an appropriate label (e.g., a class) for the unseen input. Here, unseen input is considered input that was not part of an example used in training the model. For instance, returning to the example involving the development of a machine learning model to use in predicting the likelihood of insured individuals in developing a medical condition, the trained version of the machine learning model can then be used to process unseen input representing an insured individual, and identify a label/class for the insured individual indicating whether the insured individual is likely or not to develop the medical condition. In addition to the class, the trained version of the machine learning model may be configured in some instances to provide a confidence measure (value) along with the predicted class.

As previously noted, in some domains such as finance and healthcare, security and user privacy protection oftentimes need (and sometimes are required) to be taken into consideration when developing machine learning solutions. Thus, in particular embodiments, the set of sufficiently-labeled data can serve as a layer of protection on user privacy that can be very difficult to fully penetrate. Indeed, in a set of sufficiently-labeled data {(xᵢ, xᵢ′T(yᵢ, yᵢ′))}ᵢ₌₁ⁿ, the actual values of yᵢ and yᵢ′ are oftentimes unable to be re-identified if only given T (yᵢ, yᵢ′). This can help to preserve privacy in instances when yᵢ represents sensitive information about user xᵢ. In this case, only relative information between user pairs is stored in the set of data, and absolution information on each individual user would not be recovered even in the event of a hacker obtaining access to the set of data. Accordingly, in various embodiments, this layer of encryption on the data can be provided at no cost and/or can be provided without compromise to performance. In addition, this layer of encryption can be provided in various embodiments regardless of the number of classes, contrasting some other forms of privacy-preserving labels that cease to be effective in certain cases such as binary classification. Furthermore, in particular embodiments, sufficient labels may be used as extra protection alongside many existing privacy-protecting techniques such as federated learning, homomorphic encryption, multi-party computation, anonymization, pseudonymization, and/or the like.

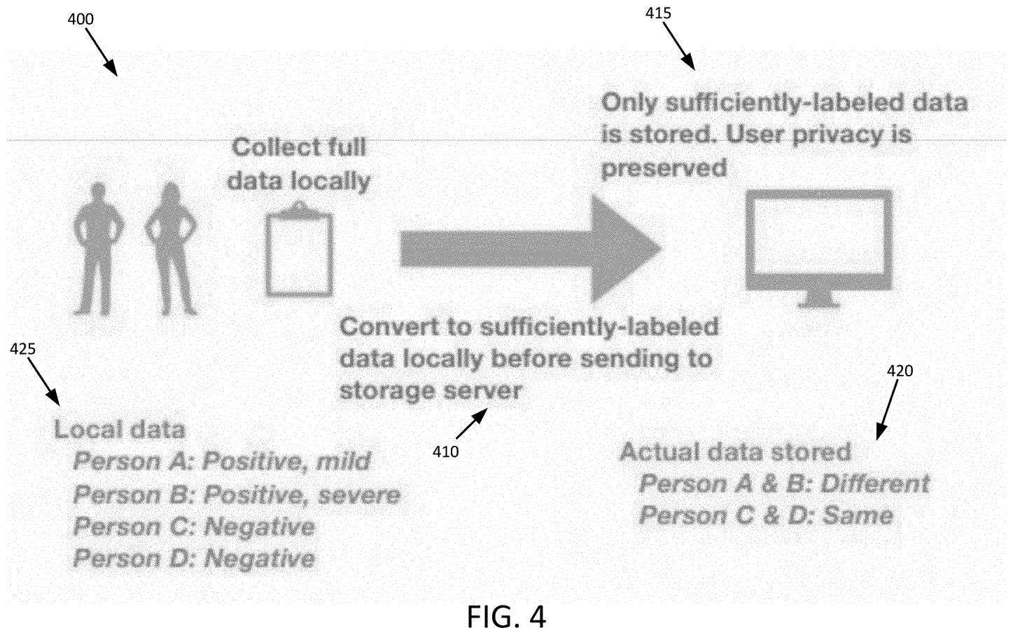

For example, a practical situation in which the privacy-preserving nature of sufficient labels generated in accordance with various embodiments can be useful is in training a predictor on whether a person has contracted an infectious disease. Whether a person has contracted the disease can be viewed as sensitive information and to protect user privacy, a hospital may convert a set of fully-labeled user data locally upon collection before transporting and/or storing this data to/on a storage server and/or medium. This pipeline 400 is demonstrated in FIG. 4. Accordingly, as a result, a hacker who may gain access to the produced set of sufficiently-labeled data during a time the data is being communicated 410 and/or once the data has been stored on the storage server and/or medium 415 is only likely to know at most if any two individuals have identical diagnosis results 420. However, the hacker is not likely to know the actual diagnosis result 425 of any individual.

### Exemplary Model Training Operations

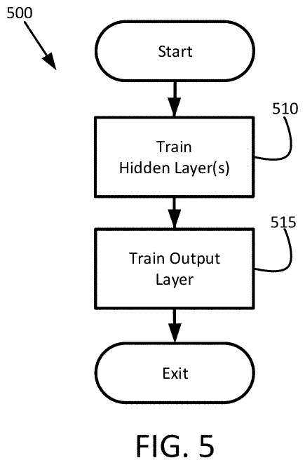

Turning now to FIG. 5, additional details are provided regarding a process flow 500 for training a machine learning model according to various embodiments. FIG. 5 is a flow diagram showing a training application for performing such functionality according to various embodiments of the disclosure. The process flow 500 may accordingly be an example embodiment of Step/Operation 315 at which the machine learning model is trained. The flow diagram shown in FIG. 5 may correspond to operations carried out by a processing element 210 in a computing entity 200, such as an application server 110 and/or an end user device 135 described in FIG. 1, as it executes the training application stored in the computing entity's volatile and/or nonvolatile memory.

Generally, the machine learning model being trained may be considered in the form f=f₂∘F₁, where [symbol] ₁, ∈F₁: [symbol] → [symbol] ^{p}, [symbol] ₂∈f₂: [symbol] ^{p}→ [symbol] . [symbol] ₁, [symbol] ₂ are some hypothesis spaces. Equation 5 provides a definition of said hypothesis spaces.

[symbol]

={f|f=f₂∘F₁,f₂∈ [symbol] ₂,F₁∈ [symbol] ₁}

### Equation 5

In addition, f₂ may be considered as a linear model in a real inner product space: f₂(⋅)= [symbol] w,ϕ(⋅) [symbol] where ϕ is some feature map, and that it is parameterized by w. In various examples, the feature map ϕ may be assumed to satisfy Equation 6.

∥ϕ(u)∥=r,∀u

### Equation 6

Thus, this model formulation remains accurate for a wide range of different popular classifier machine learning models. For example, this model formulation is broad enough to include neural networks such as the VGG networks, ResNets, DenseNets, and/or the like. For these models, ϕ can represent the nonlinearity between the model body and the final linear module. Equation 6 can be satisfied by normalizing the model activation vector after this nonlinearity.

In various embodiments, training of the machine learning model using this model formulation may involve the regular hinge loss, which is given by Equation 7. The unbounded (from below) version, given by Equation 8, may also be considered.

[symbol]

⁰⁺: [symbol] ×{+,−}→ [symbol] :( [symbol] , [symbol] ) [symbol] max(0,1− [symbol] [symbol] )

### Equation 7

[symbol] : [symbol] ×{+,−}→ [symbol] :( [symbol] , [symbol] ) [symbol] −

[symbol]

[symbol]

### Equation 8

In various embodiments, the goal during training the machine learning model includes the minimization of a risk defined according to Equation 9, the risk involving the hinge loss. A sample mean estimation of the risk can be given by Equation 10.

$$R ( {f}_{2} ∘ {F}_{1} , X , Y ) = {E}_{X , Y} ℓ ( {f}_{2} ∘ {F}_{1} ( X ) , Y ) \qquad Equation 9$$

$$\hat{R} ( {f}_{2} ∘ {F}_{1} , {\{ ( {x}_{i} , {y}_{i} ) \}}_{i = 1}^{n} ) = \frac{1}{n} {\sum }_{i = 1}^{n} ℓ ( {f}_{2} ∘ {F}_{1} ( {x}_{i} , {y}_{i} ) \qquad Equation 10$$

In Equation 10, {(xᵢ, yᵢ)}ᵢ₌₁ⁿ represents a realization of an independent and identically distributed random sample sharing the same distribution as (X, Y).

Suppose the hypothesis space [symbol] is given. Also, suppose training data {(x₁_{ᵢ},x₁_{ᵢ}′,T(y₁_{ᵢ},y₁_{ᵢ}′))}ᵢ₌₁ⁿ^{₂}, with (xᵢ_{ⱼ}, yᵢ_{ⱼ}) and (x₁_{ⱼ}′, y₁_{ⱼ}′) is given and being independent and identically distributed sharing the same distribution as (X, Y) for all i, j. Then, Equation 11 may be defined, and further, Equation 12 defines a sample mean estimation.

$${ℓ}_{1} ( {F}_{1} ( x ) , {F}_{1} ( {x}^{′} ) , T ( y , {y}^{′} ) ) = {( - 1 )}^{T ( y , {y}^{′} ) + 1} ‖ ϕ ∘ {F}_{1} ( x ) - ϕ ∘ {F}_{1} ( {x}^{′} ) ‖ \qquad Equation 11$$

$${\hat{R}}_{1} ( {F}_{1} , {\{ ( {x}_{{1}_{i}} , {x}_{{1}_{i}}^{′} , T ( {y}_{{1}_{i}} , {y}_{1 i}^{′} ) ) \}}_{i = 1}^{{n}_{1}} ) = \frac{1}{{n}_{1}} {\sum }_{i = 1}^{{n}_{1}} {ℓ}_{1} ( {F}_{1} ( {x}_{{1}_{i}} ) , {F}_{1} ( {x}_{{1}_{i}}^{′} ) , T ( {y}_{1} , {y}_{1 i}^{′} ) ) \qquad Equation 12$$

Therefore, with the machine learning model being fundamentally represented and a risk being defined, the process flow 500 may begin in various embodiments with the training application training the hidden module of the model in Step/Operation 510. In various embodiments, the hidden module includes one or more hidden layers. Accordingly, in particular embodiments, the training application trains the hidden module to find an \hat{F}₁. This training is given by Equation 13.

$${argmin}_{{F}_{1} ∈ {𝔽}_{1}} {\hat{R}}_{1} ( {F}_{1} , {\{ ( {x}_{{1}_{i}} , {x}_{{1}_{i}}^{′} , T ( {y}_{{1}_{i}} , {y}_{{1}_{i}}^{′} ) ) \}}_{i = 1}^{{n}_{1}} ) \qquad Equation 13$$

Thus, in these particular embodiments, the training application primarily makes use of the set of sufficiently-labeled data in training the hidden module. Once the hidden module has been trained, the training application trains the output module of the model in Operation 515. Here, in particular embodiments, the training application trains the output module to find an \hat{f}₂ using Equation 14.

$${argmin}_{{f}_{2} ∈ {𝔽}_{2}} \hat{R} ( {f}_{2} ∘ {\hat{F}}_{1} , {\{ ( {x}_{{2}_{i}} , {y}_{{2}_{i}} ) \}}_{i = 1}^{{n}_{2}} ) \qquad Equation 14$$

Accordingly, in these particular embodiments, the training application primarily makes use of the set of fully-labeled data in training the output module. In addition, the training application in particular embodiments is configured to keep F₁ frozen at \hat{F}₁ during training of the output module. That is to say, in these particular embodiments, the hidden representations that were learned during the training of the hidden module are kept frozen (unchanged) during the training of the output module. At the conclusion of training the output module, the training application returns \hat{f}₂∘\hat{F}₁.

Here, in various embodiments, the training application is able to find a minimizer for the true risk R given enough training data. Specifically, embodiments of the framework are configured to find, during training of the hidden module, an F₁∈ [symbol] ₁ for which there exists an f₂∈ [symbol] ₂ such that f₂∘F₁ is a risk minimizer. To demonstrate such, suppose empirical risk minimization (ERM) on \hat{R} (learning with only fully-labeled data) can map training data {(xᵢ, yᵢ)}ᵢ₌₁ⁿ and a hypothesis space [symbol] to a solution F∈ [symbol] that attains at most y true risk. Equations 15-19 may then be defined.

$$𝔻 = {argmin}_{{F}_{1} ∈ {𝔽}_{1}} {E}_{X , {X}^{′} \vert Y \neq {Y}^{′}} ( - ‖ ϕ ∘ {F}_{1} ( X ) - ϕ ∘ {F}_{1} ( {X}^{′} ) ‖ ) \qquad Equation 15$$

$$𝕊 = \{ {F}_{1} ∈ {𝔽}_{1} \vert P r ( ϕ ∘ {F}_{1} ( X ) = ϕ ∘ {F}_{1} ( {X}^{′} ) \vert Y = {Y}^{′} ) = P r ( ϕ ∘ {F}_{1} ( X ) \neq ϕ ∘ {F}_{1} ( {X}^{′} ) \vert Y \neq {Y}^{′} ) = 1 \} \qquad Equation 16$$

$${𝔽}^{*} = \{ {f}_{2} ∘ {F}_{1} \vert {f}_{2} ∘ {F}_{1} ∈ {argmin}_{{f}_{2} ∈ {𝔽}_{2} , {F}_{1} ∈ {𝔽}_{1}} R ( {f}_{2} ∘ {F}_{1} , X , Y ) \} \qquad Equation 17$$

$$If 𝔻 ⋂ 𝕊 \neq ∅ and \vert supp ϕ ∘ {F}_{1} ( X ) \vert > 4 , ∀ {F}_{1} ∉ 𝕊 , \\ then {𝔽}_{1}^{*} = 𝔻 ⋂ 𝕊 . \qquad Equation 18$$

### Equation 19

To show that embodiments of the framework can find a true risk minimizer, the training of the hidden module F₁ can be shown to find an element in [symbol] ∩ [symbol] , given enough data. To see this, Equation 20 below can be seen as an approximation to Equation 21, and Equation 22 similarly to Equation 23.

$$\frac{1}{{n}_{1}} {\sum }_{i = 1}^{{n}_{i}} ( T ( {y}_{{1}_{i}} , {y}_{{1}_{i}}^{′} ) = 0 ) ( - ‖ ϕ ∘ {F}_{1} ( {x}_{{1}_{i}} ) - ϕ ∘ {F}_{1} ( {x}_{{1}_{i}}^{′} ‖ ) \qquad Equation 20$$

$${E}_{X , {X}^{′} \vert Y \neq {Y}^{′}} ( - ‖ ϕ ∘ {F}_{1} ( X ) - ϕ ∘ {F}_{1} ( {X}^{′} ) ‖ ) \qquad Equation 21$$

$$\frac{1}{{n}_{1}} {\sum }_{i = 1}^{{n}_{1}} ( T ( {y}_{{1}_{i}} {y}_{{1}_{i}}^{′} ) = 1 ) ( ‖ ϕ ∘ {F}_{1} ( {x}_{{1}_{i}} ) - ϕ ∘ {F}_{1} ( {x}_{{1}_{i}}^{′} ‖ ) \qquad Equation 22$$

$${E}_{X , {X}^{′} \vert Y = {Y}^{′}} ‖ ϕ ∘ {F}_{1} ( X ) - ϕ ∘ {F}_{1} ( {X}^{′} ) ‖ ) \qquad Equation 23$$

Equation 23 is minimized if and only if ϕ∘F₁(X)=ϕ∘F₁(X′) w.p. 1given Y 32 Y′.

Further, it can be shown that the set of sufficiently-label data is a sufficient statistic for parameter f₂. Considering [symbol] ₁* as a random element with distribution parameterized by f₂, the conditional distribution of [symbol] ₁*, given (X, X′, T(Y, Y′)), does not depend on f₂. Thus, by definition of a sufficient statistic, (X, X′, T(Y, Y′)) can be concluded to be a sufficient statistic for f₂. This further suggests that (X, X′, T(Y, Y′)) contains all the information of (X, Y) when it comes to finding an optimal classifier.

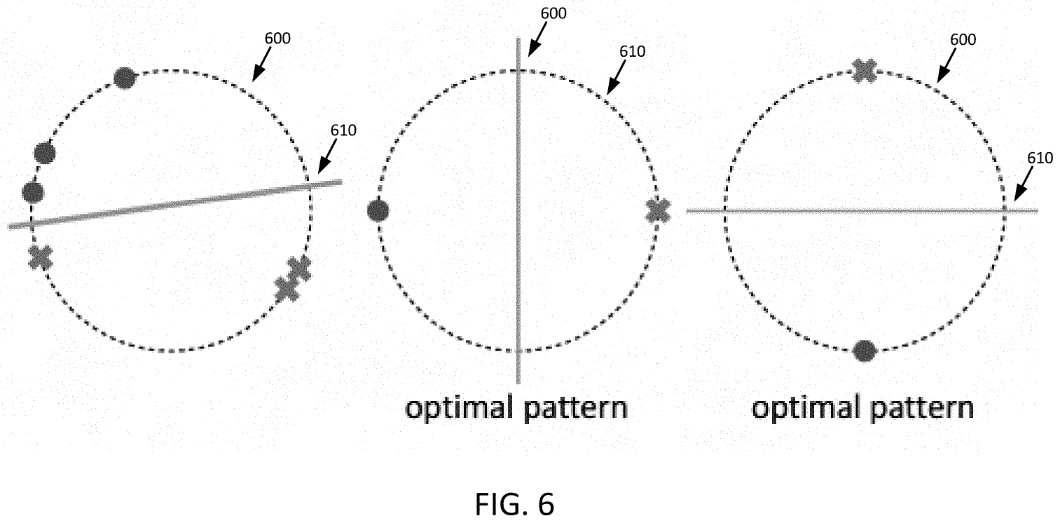

In classification, data examples are usually considered individually and used with full label information. However, various embodiments of the framework make use of pairwise representations of data that contain all relevant information for learning the optimal hidden representations, and can allow for data reduction. Turning to FIG. 6, consider the hidden module F₁ to be learning a pattern in a feature space such that the output module f₂, e.g., a linear classifier in that feature space, can most effectively classify (in terms of minimizing the hinge loss). Due to the assumption on the feature map, i.e., ∥ϕ(u)∥−r, ∀u, every example must be on a circle 600 as illustrated in FIG. 6. For each pattern, the output module f₂ that achieves zero hinge loss (perfect separation) with minimum ∥w∥ (model capacity) is the line 610. The weight of the line 610 is proportional to ∥w∥.

The optimal pattern that allows perfect separation with the smallest possible capacity (and therefore best generalization), is the one where each pair of examples from the same class are mapped to the same point, whereas each pair from different classes are as far away as possible. Therefore, as illustrated in FIG. 6, how the hidden module F₁ arranges each individual example does not matter, all patterns are equally optimal if (1) each pair from distinct classes are mapped as far away from each other as possible and (2) each pair from the same class are mapped to the same point. This pattern is fully described using pairwise summary and that an optimal hidden module F₁ is one that learns such a pattern is essentially what is shown via Equations 15-23.

Accordingly, this sufficiency of (X, X′, T(Y, Y′)) can enabling partitioning of the sample space and leave only relevant information. Thus, various embodiments of the framework enable learning with sufficiently-labeled example-pairs directly using only relevant data. In addition, obtaining sufficiently-labeled data from fully-labeled data can be interpreted as extracting relevant information prior to any training.

Finally, from the perspective of learning representations, ϕ∘F₁ determines the learned internal representations of a model. Therefore, various embodiments of the framework enable internal representations for classification to be learned using only sufficiently-labeled data. Such a configuration can provide an advantage in that fully-labeled data is only needed to find the optimal linear mapping into the label space.

Furthermore, various embodiments of the framework enable learning with sufficiently-labeled data as efficiently as learning with fully-labeled data in terms of the number of labeled training examples needed. In other words, various embodiments of the framework can find a solution with a certain test performance using a mixture of sufficiently-labeled and fully-labeled data, with a total sample size comparable to a sample of n fully-labeled examples used by ERM to find a solution with a similar test performance.

First, it is a standard result that the sample complexity of learning, when formulated as the maximum absolute difference between the empirical risk (sample mean estimation of the risk) and the true risk over some hypothesis class, can be bounded with the sum of a [symbol] (1/\sqrt{n}) term and a (fixed) scalar multiple of a complexity measure such as the Gaussian complexity on the underlying hypothesis class composed with the loss function, where n is the size of the training sample on which the empirical risk is evaluated.

The Gaussian complexity of a set [symbol] of functions, each mapping from some space [symbol] into [symbol] , is defined by Equation 24.

$${𝒢}_{n} ( ℍ ) = E {sup}_{h ∈ ℍ} \frac{1}{n} {\sum }_{i = 1}^{n} {g}_{i} h ( {u}_{i} ) \qquad Equation 24$$

In Equation 24, {uᵢ}ᵢ₌₁ⁿ represents a set of independently and identically distributed random elements defined on [symbol] , and {gᵢ}ᵢ₌₁ⁿ represents a set of independently and identically distributed standard normal random variables. Equations 25-27 are then defined.

$$ℓ ∘ 𝔽 = \{ ( x , y ) ↦ ℓ ( F ( x ) , y ) \vert F ∈ 𝔽 \} \qquad Equation 25$$

$${ℓ}_{1} ∘ {𝔽}_{1} = \{ ( x , {x}^{′} , T ( y , {y}^{′} ) ↦ {ℓ}_{1} ( F ( x ) , F ( {x}^{′} ) , T ( y , {y}^{′} ) ) \vert F ∈ {𝔽}_{1} \qquad Equation 26$$

$$ℓ ∘ {𝔽}_{2} ∘ {F}_{1} = \{ ( x , y ) ↦ ℓ ( {f}_{2} ∘ {F}_{1} ( x ) , y ) \vert {f}_{2} ∈ {𝔽}_{2} \} , \\ for some fixed {F}_{1} \qquad Equation 27$$

Accordingly, the Gaussian complexity of [symbol] ₁∘ [symbol] ₁ is similar (in terms of the speed of convergence in n)² to that of [symbol] ∘ [symbol] , and the Gaussian complexity of [symbol] ₁∘ [symbol] ₂∘F₁ is [symbol] (1/\sqrt{n}) for any F₁. This then demonstrates that ERM using fully-labeled data has similar sample complexity as using sufficiently-labeled data in various embodiments.

For instance, suppose Equation 28, in which [symbol] _{1,i} is a set of functions mapping from [symbol] into [symbol] for all i, and Equation 29 for some A>0.

[symbol]

₁={F₁:u [symbol] (f_{1,1}(u), . . . , f_{1,p}(u))^{τ}|f_{1,i}∈ [symbol] _{1,i},∀i}

### Equation 28

[symbol] ₂={f₂(⋅)= [symbol] w,ϕ(⋅) [symbol] |∥w∥≤A}

### Equation 29

With an assumption that ϕ is ρ-Lipschitz with respect to the Euclidean metric on [symbol] ^{p}, then Equation 30 can be defined.

$${𝒢}_{n} ( ℓ ∘ 𝔽 ) \leq 4 A ρ {\sum }_{i = 1}^{p} {𝒢}_{n} ( {𝔽}_{i , 1} ) \qquad Equation 30$$

As a further result of the Gaussian complexity demonstration, suppose Equation 31. In Equation 31, [symbol] _{1,i} is a set of functions mapping from [symbol] into [symbol] for all i.

[symbol]

₁={F₁:u [symbol] (f_{1,1}(u), . . . , f_{1,p}(u))^{τ}|f_{1,i}∈ [symbol] _{1,i},∀i}

### Equation 31

With an assumption that ϕ is p-Lipschitz with respect to the Euclidean metric on [symbol] ^{p}, then Equation 32 can be defined.

$${𝒢}_{n} ( {ℓ}_{1} ∘ {𝔽}_{1} ) \leq 8 ρ {\sum }_{i = 1}^{p} {𝒢}_{n} ( {𝔽}_{i , 1} ) \qquad Equation 32$$

As yet another result of the Gaussian complexity demonstration, let [symbol] ₂{f²(⋅)= [symbol] w,ϕ(⋅) [symbol] |∥w∥≤A} for some A>0. Then for any F₁, Equation 33 holds.

$${𝒢}_{n} ( ℓ ∘ {𝔽}_{2} ∘ {F}_{1} ) \leq \frac{A r}{\sqrt{n}} \qquad Equation 33$$

Accordingly, these results together show that, if a training framework can use fully-labeled data to find a solution with a certain test performance, embodiments of the framework described herein can find a solution with similar test performance using a mixture of sufficiently-labeled and fully-labeled data, with total sample size being comparable to n.

In addition to various embodiments of the framework being able to find a solution with a similar test performance, embodiments of the framework can do so using a decreased number of fully-labeled examples as the number of sufficiently-labeled example-pairs increases. For instance, define [symbol] _{2,A}={f₂|∥w∥≤A}, and order these hypothesis spaces with A. Let [symbol] , [symbol] be defined as described above in Equations 15 and 16 with the assumptions therein satisfied. And let a true risk value γ<0³ be given. For each F₁∈ [symbol] ₁ such that there f₂∈ [symbol] ₂ exists with f₂∘F₁ attaining this true risk value, denote the smallest [symbol] _{2,A} such that min [symbol] R(f₂∘F₁,X,Y)=γ as [symbol] _{2,f}_{_{1,}}_{γ}. Let an n₂∈ [symbol] be given and define Equation 34.

$${\hat{𝔽}}_{2 , {F}_{1} \cdot γ}^{*} = {argmin}_{{f}_{2} ∈ {𝔽}_{2} , {F}_{1} \cdot γ} \hat{R} ( {f}_{2} ∘ {F}_{1} , {\{ ( {x}_{{2}_{i}} , {y}_{{2}_{i}} ) \}}_{i = 1}^{{n}_{2}} ) \qquad Equation 34$$

Then a nonnegative function t(F₁) and a positive constant η can be found such that for any given probability δ, Equations 35 and 36 can be defined with probability at least 1−δ.

$${sup}_{{\hat{f}}_{2} ∈ {\hat{𝔽}}_{2}^{*} , {F}_{1} ∘ γ} R ( {\hat{f}}_{2} ∘ {F}_{1} , X , Y ) - γ \leq 2 η \frac{t ( {F}_{1} ( r}{\sqrt{{n}_{2}}} + 5 t ( {F}_{1} ) r \sqrt{\frac{2 ln ( 8 / δ )}{{n}_{2}}} \qquad Equation 35$$

$${argmin}_{{F}_{1} ∈ {𝔽}_{1}} t ( {F}_{1} ) = 𝕊 ⋂ 𝔻 \qquad Equation 36$$

The above describes how the training of the hidden module F₁ affects the data requirement for training the output module f₂. In particular embodiments, a small t(F₁) results in a small n₂ for the right hand side of the inequality to stay fixed at a specific value. This indicates that for training the output module f₂, the framework in various embodiments needs the fewest fully-labeled data to produce a solution that attains a particular test performance if the training of the hidden module F₁ produce a minimizer for t(F₁), confirming that training of the hidden module F₁ conducted by the framework does produce a minimizer for t(F₁) since such embodiments finds an element in [symbol] ∩ [symbol] , given enough sufficiently-labeled data. Therefore, the more sufficiently-labeled data that the framework can leverage in performing the training of the hidden module F₁, the better such embodiments of the framework can perform in finding an element in [symbol] ∩ [symbol] , and, consequently, the fewer fully-labeled training data needed by the framework in training the output module f₂ to attain a particular test performance.

Accordingly, this result may only go in one direction for various embodiments of the framework. That is to say, for various embodiments of the framework, the training of the output module f₂ or the amount of fully-labeled examples given does not affect the data requirement of training the hidden module F₁. This is a result of the framework in various embodiments being configured to train the hidden module F₁ and the output module f₂ sequentially, with the hidden module F₁ frozen after its training.

### Additional Exemplary Embodiments of the Framework

The discussion provided herein so far has been directed to using embodiments of the framework in training a machine learning model for binary classification with the hinge loss, according to a specific loss function. However, embodiments of the framework can be extended to other machine learning models such as, for example, models used in classification applications having c classes greater than two (c≥2), and using an arbitrary loss function. For instance, the machine learning model to be trained may take the form described in Equations 37 and 38, in various examples.

F=F₂∘F₁,F₁: [symbol] → [symbol] ^{p},F₂: [symbol] ^{p}→ [symbol] ^{c}

### Equation 37

F₂:u [symbol] ( [symbol] w₁,ϕ(u) [symbol] +b₁, . . . , [symbol] w_{c},ϕ(u) [symbol] +b_{c})τ

### Equation 38

In the above equations, F₂ represents a vector of binary classifiers, each classifying one class versus the rest. The ith coordinate of F₂ corresponds to the ith class. Accordingly, the model outputs the class corresponding to the maximum coordinate of F₂. For example, many popular classification networks including ResNet neural networks admit such a representation.

Let two loss functions [symbol] : [symbol] ^{c}×{1, . . . , c}→ [symbol] , [symbol] ₁: [symbol] ^{p}× [symbol] ^{p}×{0,1}→ [symbol] be given. For example, [symbol] can be softmax followed by cross-entropy, although, [symbol] can be other classification losses. Also suppose a hypothesis space and two sets of labeled data—a set of sufficiently-labeled data and a set of fully-labeled data—with there being potentially more than two classes in the dataset for classification. In these instances, embodiments of the framework (e.g., the training application) may be configured to carrying out the training of the hidden module to find an \hat{F}₁ using Equation 39.

$${argmin}_{{F}_{1} ∈ {𝔽}_{1}} \frac{1}{{n}_{1}} {\sum }_{i = 1}^{{n}_{1}} {ℓ}_{1} ( {F}_{1} ( {x}_{{1}_{i}} ) , {F}_{1} ( {x}_{{1}_{i}}^{′} ) , T ( {y}_{{1}_{i}} , {y}_{{1}_{i}}^{′} ) ) \qquad Equation 39$$

In addition, embodiments of the framework may be configured to carrying out the training of the output module to find an [symbol] in Equation 40.

$${argmin}_{{F}_{2} ∈ {𝔽}_{2}} \frac{1}{{n}_{2}} {\sum }_{i = 1}^{{n}_{2}} ℓ ( {F}_{2} ∘ {\hat{F}}_{1} ( {x}_{{2}_{i}} ) , {y}_{{2}_{i}} ) \qquad Equation 40$$

Accordingly, the framework returns [symbol] ∘\hat{F}₁. Thus, when the number of classes is two (c=2), implementing F₂ as a real-valued function without bias, choosing [symbol] ₁ as in Equation 11, and then letting [symbol] be the unbounded binary hinge loss as previously described in Equation 8 recovers the embodiments of the framework as previously described.

In addition to the [symbol] ₁ previously described, embodiments of the framework may be configured to use other alternatives that can learn a hidden module F₁ that satisfies the requirements of finding a risk minimizer when plugged into Equation 44. For instance, in various embodiments, any [symbol] ₁ that encourages the hidden module to map examples from distinct classes further away from each other and map examples from the same class closer to each other in the feature space defined by can be used.

For example, a bivariate function k(u,v)= [symbol] ϕ(u),ϕ(v) [symbol] can be defined. Let β:=min k and define kᵢ*= [symbol] (T(y₁_{ᵢ},y₁_{ᵢ}′)=1) r²+ [symbol] (T(y₁_{ᵢ}, y₁_{ᵢ}′)=0) β. Denote the vector in [symbol] ⁿ^{₁} whose ith element is k (F₁(x₁_{ᵢ}),F₁(x₁_{ᵢ}′)) as K_{F}_{₁} and the vector whose ith element is kᵢ* as K*.

A negative cosine similarity (NCS) can be defined with Equation 41.

$${ℓ}_{1} ( {F}_{1} ( {x}_{{1}_{i}} ) , {F}_{1} ( {x}_{{1}_{i}}^{′} ) , \\ T ( {y}_{{1}_{i}} , {y}_{{1}_{i}}^{′} ) ) = - {n}_{1} \frac{k ( {F}_{1} ( {x}_{{1}_{i}} ) , {F}_{1} ( {x}_{{1}_{i}}^{′} ) ) {k}_{i}^{*}}{{{‖ {K}_{{F}_{1}} ‖}_{2} {K}^{*} ‖}_{2}} \qquad Equation 41$$

Here, when the negative cosine similarity is plugged into Equation 39, the empirical risk to be minimized becomes the negative cosine similarity between K_{F}_{₁} and K*.

In various other embodiments, a contrastive loss function as defined in Equation 42 may be used with Equation 39.

$${ℓ}_{1} ( {F}_{1} ( {x}_{{1}_{i}} ) , {F}_{1} ( {x}_{{1}_{i}}^{′} ) , \\ \\ T ( {y}_{{1}_{i}} , {y}_{{1}_{i}}^{′} ) ) = - log ( \frac{{\sum }_{i = 1}^{{n}_{1}} ( T ( {y}_{{1}_{i}} , {y}_{{1}_{i}}^{′} ) = 1 ) {e}^{k ( {F}_{1} ( {x}_{{1}_{i}} {F}_{1} ( {x}_{{1}_{i}}^{′} ) ) )}}{{\sum }_{i = 1}^{{n}_{1}} {e}^{k ( {F}_{1} ( {x}_{{1}_{i}} {F}_{1} ( {x}_{{1}_{i}}^{′} ) ) )}} \qquad Equation 42$$

As a further alternative, mean squared error as defined by Equation 43 can be used as a loss function in Equation 39.

[symbol]

₁(F₁(x₁_{ᵢ}(x₁_{ᵢ}),F₁(x₁_{ᵢ}′),T( [symbol] ₁_{ᵢ},y₁_{ᵢ}′))=(k(F₁(x₁_{ᵢ}),F₁(x₁_{ᵢ}′))−kᵢ*)²

### Equation 43

In various embodiments, the analysis provided herein may be extended to this model by, for example, noting that each of the output node is a one-class-versus-the-rest (binary) classifier.

### Sample Implementation of Various Embodiments

A discussion is now provided on example results of training machine learning models based on embodiments of the framework. Here, testing was conducted for both the hinge loss and the cross-entropy loss on the Modified National Institute of Standards and Technology (MNIST), Fashion-MNIST, Street View House Numbers (SVHN), and Canadian Institute for Advanced Research (CIFAR-10) datasets that are shown through this test (1) can train state-of-the-art classifiers with a mixture of sufficiently-labeled and fully-labeled data, and (2) can do so, only needing a small set of fully-labeled data if given enough sufficiently-labeled data.

The settings during the test for training the models are as follows. A LeNet-5 convolutional neural network model is trained for MNIST dataset and a ResNet-18 convolutional neural network model is trained for Fashion-MNIST, SVHN, and CIFAR-10 datasets. To ensure that the models satisfied the assumption described herein, the activation vector of the nonlinearity before the final linear module was always normalized to one by (elementwise) dividing itself by its norm. This normalization did not affect performance. The optimizer used was stochastic gradient descent with batch size 128. Each hidden module (or full model, in the cases where only full labels were used) was trained with step size 0.1, 0.01, and 0.001, for 200, 100, and 50 epochs, respectively. If needed, each output module was trained with step size 0.1 for 50 epochs. For MNIST, the data was preprocessed by training set sample mean subtraction and then divided by training set sample standard deviation. For the Fashion-MNIST, SVHN, and CIFAR-10 datasets, the data was randomly cropped and flipped after the said normalization procedure. Training examples of size 5 k/10 k/20 k/5 k/10 k/20 k/5 k/10 k/20 k were randomly selected to form the validation set for a training set of size 50 k/100 k/200 k/60 k/120 k/240 k/73,257/146,514/293,028. The validation set was used for tuning hyperparameters and for determining the best model to save during each training session. For a training set of size 10, no validation data was used, and the optimal model was chosen based on the convergence of the loss function value on the training data. For the SVHN dataset, the “additional” training images where not used. For all datasets, performance is reported on the standard test sets. None of the proposed [symbol] ₁'s were found to significantly outperform the others. In all cases, performance is reported using NCS.

In obtaining a set of sufficiently-labeled data, all of these curated datasets that were selected are fully-labeled, and the sets of sufficiently-labeled data used were derived from fully-labeled data. For example, to create a set of sufficiently-labeled data with size 100 k, 100 k pairs of examples were randomly sampled from the original fully-labeled training set. And for each pair, a sufficient label of 1 or 0 was generated based on their original full labels. Some pairs that did not contain useful information were discarded. Specifically, suppose the input examples in the original dataset are {xᵢ}ᵢ₌₁ⁿ, then the sampled set of example-pairs is {xᵢ, x₂}ᵢ₌₁ⁿ^{₁} with 1ᵢ<2ᵢ and (1ᵢ, 2ᵢ), ≠(1ⱼ, 2ᵢ), ∀ᵢ≠j. Such a set of sufficiently-labeled data covers nᵢ/(n(n−1)/2) of all possible informative example-pairs from the original training dataset.

Accordingly, an online random sampling strategy (“online”) is used to approximate the performance upper bound for training with sufficiently-labeled data using these curated datasets. Namely, in each training epoch, the original fully-labeled dataset was iterated in batches of size 128 and for each batch of fully-labeled data, and converted into a set of sufficiently-labeled pairs (by taking pairwise combinations, with uninformative pairs discarded as before) and used to compute a model update step. Thus, since the dataset was randomly shuffled at each epoch, all sufficiently-labeled pairs can potentially be exhausted given enough training epochs. In “online,” the size of the validation set was chosen based on the size of the original fully-labeled dataset using the rules above. The results of the test are shown in Table 1:

TABLE 1

| | | Date Usage | | Test Acc. |
|---|---|---|---|---|
| Dataset (Model Used) | Loss | Full | Suff. | (%) |
| MNIST (LeNet-5) | Hinge | 60k | 0 | 99.22 ± 0.05 |
| | | 10 | 120k | 98.97 ± 0.15 |
| | | 10 | 240k | 99.12 ± 0.08 |
| | | 10 | online | 99.23 ± 0.05 |
| | Cross-Entropy | 60k | 0 | 99.32 ± 0.08 |
| | | 10 | 120k | 98.98 ± 0.15 |
| | | 10 | 240k | 99.12 ± 0.07 |
| | | 10 | online | 99.23 ± 0.05 |
| Fashion-MNIST (ResNet- | Hinge | 60k | 0 | 95.11 ± 0.12 |
| 18) | | 10 | 120k | 93.85 ± 0.29 |
| | | 10 | 240k | 94.61 ± 0.17 |
| | | 10 | online | 95.03 ± 0.28 |
| | Cross-Entropy | 60k | 0 | 95.10 ± 0.18 |
| | | 10 | 120k | 93.89 ± 0.32 |
| | | 10 | 240k | 94.63 ± 0.15 |
| | | 10 | online | 95.03 ± 0.27 |
| SVHN (ResNet-18) | Hinge | 73, 257 | 0 | 96.49 ± 0.07 |
| | | 10 | 146, 514 | 95.77 ± 0.12 |
| | | 10 | 293, 028 | 96.11 ± 0.11 |
| | | 10 | online | 96.06 ± 0.16 |
| | Cross-Entropy | 73, 257 | 0 | 96.46 ± 0.10 |
| | | 10 | 146, 514 | 95.78 ± 0.11 |
| | | 10 | 293, 028 | 96.13 ± 0.10 |
| | | 10 | online | 96.67 ± 0.16 |
| CIFAR (ResNet-18) | Hinge | 50k | 0 | 94.09 ± 0.12 |
| | | 10 | 100k | 90.99 ± 0.33 |
| | | 10 | 200k | 93.56 ± 0.24 |
| | | 10 | online | 94.24 ± 0.25 |
| | Cross-Entropy | 50k | 0 | 94.19 ± 0.13 |
| | | 10 | 100k | 91.00 ± 0.34 |
| | | 10 | 200k | 93.63 ± 0.17 |
| | | 10 | online | 94.24 ± 0.24 |

For “Data Usage” in the table, “Full” refers to the number of fully-labeled examples, and “Suff” refers to the number of sufficiently-labeled example-pairs. The models that were trained without the use of sufficiently-labeled example-pairs were trained with standard end-to-end backpropagation. For the models that used ten fully-labeled examples, these examples were randomly selected at each trial, with one example from each class.

Accordingly, the results provided in Table 1 demonstrate that various embodiments of the framework can train state-of-the-art classifiers using almost only sufficiently-labeled data. Here, the results show that embodiments of the framework were able to sufficiently train the models using a single randomly-chosen fully-labeled example from each class. Further, the results demonstrate various embodiments of the framework can achieve similar test performance when using a training dataset of a mixture of sufficiently-labeled and fully-labeled data with a size similar to that of a training dataset required in training a model using only fully-labeled data. Thus, the results show that embodiments of the framework can enjoy similar sample complexity as training frameworks that make use of only fully-labeled data.

### CONCLUSION

Many modifications and other embodiments of the disclosure set forth herein will come to mind to one skilled in the art to which these modifications and other embodiments pertain having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A method for training a machine learning model comprising a hidden module and an output module and configured for predicting one of a plurality of original labels for an input, the method comprising:
generating, via one or more processors, sufficiently-labeled data comprising a plurality of example-pairs, wherein each example-pair is associated with a sufficient label indicating whether a first input example and a second input example of the example-pair are identified as having a same original label from the plurality of original labels;
training, via one or more processors, the hidden module of the machine learning model using the sufficiently-labeled data;
sequentially after the training of the hidden module of the machine learning model, training, via the one or more processors, the output module of the machine learning model using a plurality of input examples each having one of the plurality of original labels to generate a trained machine learning model; and
automatically providing the trained machine learning model for use in one or more prediction tasks.

2. The method of claim 1, wherein the plurality of input examples used in training the output module are obtained from fully-labeled data used to generate the sufficiently-labeled data.

3. The method of claim 1, wherein the trained machine learning model is configured to, for the one or more prediction tasks, identify an original label from the plurality of original labels for an unseen input provided to the trained machine learning model.

4. The method of claim 1, wherein the plurality of original labels comprises a first original label classifying an individual as contracting a disease and a second original label classifying the individual as not contracting the disease, and wherein the one or more prediction tasks includes identification of either the first original label or the second original label for an unseen individual to indicate a likelihood of the unseen individual having contracted the disease.

5. The method of claim 1, wherein generating sufficiently-labeled data comprises:
obtaining fully-labeled data comprising a plurality of input examples each having one of the plurality of original labels;
generating a plurality of example-pairs, each example-pair comprising the first input example selected from the fully-labeled data and the second input example selected from the fully-labeled data; and
generating a sufficient label for each of the plurality of example-pairs based at least in part on summarizing each original label of a respective first input example and a respective second input example.

6. The method of claim 5, wherein the sufficient label for each example-pair is generated using an annotation machine learning model.

7. The method of claim 1, further comprising storing the sufficiently-labeled data in a storage medium as an encrypted representation of the first input example and the second input example.

8. The method of claim 1, wherein hidden representations that were learned during the training of the hidden module are kept unchanged during the training of the output module.

9. The method of claim 1, wherein the machine learning model comprises a neural network configured as a classifier, and each original label of the plurality of original labels comprises a class.

10. The method of claim 1, wherein the hidden module is trained using one of a hinge loss function, a negative cosine similarity function, a contrastive function, or a mean squared error.

11. An apparatus for training a machine learning model comprising a hidden module and an output module and configured for predicting one of a plurality of original labels for an input, the apparatus comprising:
at least one processor;
at least one memory including program code, wherein the at least one memory and the program code are configured to, with the at least one processor, cause the apparatus to at least:
generate sufficiently-labeled data comprising a plurality of example-pairs, wherein each example-pair is associated with a sufficient label indicating whether a first input example and a second input example of the example-pair are identified as having a same original label from the plurality of original labels;
train the hidden module of the machine learning model using the sufficiently-labeled data;
sequentially after the training of the hidden module of the machine learning model, train the output module of the machine learning model using a plurality of input examples each having one of the plurality of original labels to generate a trained machine learning model; and
automatically provide the trained machine learning model for use in one or more prediction tasks.

12. The apparatus of claim 11, wherein the trained machine learning model is configured to, for the one or more prediction tasks, identify an original label from the plurality of original labels for an unseen input provided to the trained machine learning model.

13. The apparatus of claim 11, wherein the plurality of original labels comprises a first original label classifying an individual as contracting a disease and a second original label classifying the individual as not contracting the disease, and wherein the one or more prediction tasks includes identification of either the first original label or the second original label for an unseen individual to indicate a likelihood of the unseen individual having contracted the disease.

14. The apparatus of claim 11, wherein generating the sufficiently-labeled data comprises:
obtaining fully-labeled data comprising a plurality of input examples each having one of the plurality of original labels;
generating a plurality of example-pairs, each example-pair comprising the first input example selected from the fully-labeled data and the second input example selected from the fully-labeled data; and
generating a sufficient label for each of the plurality of example-pairs based at least in part on summarizing each original label of a respective first input example and a respective second input example.

15. The apparatus of claim 14, wherein the sufficient label for each example-pair is generated using an annotation machine learning model.

16. The apparatus of claim 11, further comprising storing the sufficiently-labeled data in a storage medium as an encrypted representation of the first input example and the second input example.

17. The apparatus of claim 11, wherein hidden representations that were learned during the training of the hidden module are kept unchanged during the training of the output module.

18. The apparatus of claim 11, wherein the machine learning model comprises a neural network configured as a classifier, and each original label of the plurality of original labels comprises a class.

19. A non-transitory computer storage medium for training a machine learning model comprising a hidden module and an output module and configured for predicting one of a plurality of original labels for an input, the non-transitory computer storage medium comprises instructions configured to cause one or more processors to at least perform operations configured to:
generate sufficiently-labeled data comprising a plurality of example-pairs, wherein each example-pair is associated with a sufficient label indicating whether a first input example and a second input example of the example-pair are identified as having a same original label from the plurality of original labels;
train the hidden module of the machine learning model using the sufficiently-labeled data;
sequentially after the training of the hidden module of the machine learning model, train the output module of the machine learning model using a plurality of input examples each having one of the plurality of original labels to generate a trained machine learning model; and
automatically provide the trained machine learning model for use in one or more prediction tasks.

20. The non-transitory computer storage medium of claim 19, wherein generating the sufficiently-labeled data comprises:
obtaining fully-labeled data comprising a plurality of input examples each having one of the plurality of original labels;
generating a plurality of example-pairs, each example-pair comprising the first input example selected from the fully-labeled data and the second input example selected from the fully-labeled data; and
generating a sufficient label for each of the plurality of example-pairs based at least in part on summarizing each original label of a respective first input example and a respective second input example.
